(19) United States
(12) Patent Application Publication
(10) Pub. No.: US 2025/0344990 A1
(43) Pub. Date: Nov. 13, 2025

(54) VISUAL ELECTROPHYSIOLOGY DEVICE

(71) Applicant: LKC TECHNOLOGIES, INC., Gaithersburg, MD (US)

(72) Inventors: Charles Quentin DAVIS, Gaithersburg, MD (US); Richard COOK, Gaithersburg, MD (US)

(21) Appl. No.: 18/867,569

(22) PCT Filed: May 25, 2023
(86) PCT No.: PCT/US2023/023456

§ 371 Date: Nov. 20, 2024

Related U.S. Application Data
(60) Provisional application No. 63/345,528, filed on May 25, 2022.

(51) Int. Cl. A61B 5/398; A61B 3/00

(52) U.S. Cl. CPC A61B 5/398; A61B 3/0008; A61B 2560/0252

(57) ABSTRACT

Visual system function can be assessed using devices and methods that monitor the electrical response to a visual stimulus. Improvements in stimulus generation disclosed herein can be used separately or in combination, including improvements in wavelength accuracy, luminance accuracy, and safety. Improvements in wavelength accuracy may be accomplished by reducing the temperature range experienced by a light emitter that creates the visual stimulus. Improvements in luminance accuracy may be accomplished by reducing the temperature range experienced by the light emitter, reducing (or computationally correcting for) the temperature range experienced by a light detector used to calibrate the light output or to stop the light output after a target value is reached. Safety improvements include having an independent circuit to detect and limit the light output to levels below a target value.

### CROSS REFERENCE TO RELATED APPLICATIONS

This application claims priority to U.S. Provisional Patent Application No. 63/345,528, filed May 25, 2022, the entire contents of which are incorporated herein by reference.

### BACKGROUND OF THE INVENTION

### 1. Field of the Invention

The embodiments described herein relate to improved devices and methods for assessing visual system function.

### 2. Description of Related Art

The electroretinogram (ERG) and visual evoked potentials (VEP) are diagnostic tests used to help assess visual system function. See, for example, the textbook Principles and Practice of Clinical Electrophysiology of Vision, 2^{nd} edition, edited by Heckenlively and Arden (2006), which describes dozens of diseases that can be diagnosed with the aid of visual electrophysiology. Standards have been developed for the most common of these tests, as described in Robson et al. (2022), Hoffmann et al. (2021), Bach et al. (2012), and Odom et al. (2016). As a specific example, some features of the clinical ERG are strongly correlated with diabetic retinopathy (Bresnick and Palta (1987), Han and Ohn (2000) and Satoh et al. (1994)). As another example, Kjeka et al. (2013) showed greatly improved outcomes for the treatment of central retinal vein occlusion when basing treatment decisions on ERG results rather than ophthalmologic examinations alone.

The inventions described in U.S. Pat. Nos. 7,540,613, 9,492,098, and 9,931,032 represent the state-of-the-art in visual electrophysiology devices. Nevertheless, there still exists a need for visual electrophysiology devices that have improved performance.

### SUMMARY

Described herein are embodiments of a device and method for providing an indication of visual system function. The improvements in stimulus generation disclosed herein can be used separately or in combination, including improvements in wavelength accuracy, luminance accuracy, and safety.

In accordance with an embodiment of a device that provides an indication of visual system function of a patient, the device has an emitter capable of emitting visible light, an optical assembly arranged so that light emitted from the emitter reaches an eye of the patient; and a controller. The controller is configured to modulate a light emission from the emitter to create a light stimulus, receive and analyze an electrical signal from the visual system of the patient to create an analysis, and provide an indication of visual system function based on the analysis. The device may also include an active thermal control system configured to reduce temperature variability near the emitter. Alternatively, the device may also include a light detector arranged to detect light from the emitter and a temperature sensor configured to detect a temperature near the light detector. Alternatively, the device may also include a circuit that limits a time-averaged light from the emitter in a fashion that is independent from the controller when the emitter can generate a continuous light emission. Alternatively, the device may include a light detector arranged to detect light from the emitter and a control circuit that modulates, when the light stimulus comprises one or more flashes of light, a duration of each flash of light based on an output from the light detector obtained during that flash of light.

In some embodiments, the device includes more than one of the above-mentioned alternatives. For example, the device may include both an active thermal control system configured to reduce temperature variability near the emitter, and a light detector arranged to detect light from the emitter and a temperature sensor configured to detect a temperature near the light detector. As another example, the device may include an active thermal control system configured to reduce temperature variability near the emitter, an emitter that can generate a continuous light emission, and a circuit that limits a time-averaged light from the emitter in a fashion that is independent from the controller. As another example, the device may include a light detector arranged to detect light from the emitter and a temperature sensor configured to detect a temperature near the light detector, an emitter that can generate a continuous light emission, and a circuit that limits a time-averaged light from the emitter in a fashion that is independent from the controller. As another example, the device may include an active thermal control system configured to reduce temperature variability near the emitter, an emitter that can generate a continuous light emission, and a circuit that limits a time-averaged light from the emitter in a fashion that is independent from the controller, a light detector arranged to detect light from the emitter and a temperature sensor configured to detect a temperature near the light detector, and a circuit that limits a time-averaged light from the emitter in a fashion that is independent from the controller. As another example, the light stimulus comprises one or more flashes of light and the device may include a light detector arranged to detect light from the emitter, a temperature sensor configured to detect a temperature near the light detector, and a control circuit that modulates a duration of each flash of light based on an output from the light detector obtained during that flash of light.

In accordance with another embodiment of a device that provides an indication of visual system function of a patient, the device has an emitter capable of emitting visible light, an optical assembly arranged so that when in use light emitted from the emitter reaches an eye of the patient, and a controller. The controller when in use modulates a light emission from the emitter to create a light stimulus, receives and analyses an electrical signal from the visual system of the patient to create an analysis, and provides an indication of visual system function based on the analysis. The device includes an active thermal control system configured to reduce temperature variability near the emitter.

In accordance with another embodiment of a device that provides an indication of visual system function of a patient, the device has an emitter capable of emitting visible light, an optical assembly arranged so that when in use light emitted from the emitter reaches an eye of the patient, and a controller. The controller when in use modulates a light emission from the emitter to create a light stimulus, receives and analyses an electrical signal from the visual system of the patient to create an analysis, and provides an indication of visual system function based on the analysis. The device includes a light detector arranged to detect light from the emitter and a temperature sensor configured to detect a temperature near the light detector.

In accordance with another embodiment of a device that provides an indication of visual system function of a patient, the device has an emitter capable of emitting visible light, an optical assembly arranged so that when in use light emitted from the emitter reaches an eye of the patient, and a controller. The controller when in use modulates a light emission from the emitter to create a light stimulus, receives and analyses an electrical signal from the visual system of the patient to create an analysis, and provides an indication of visual system function based on the analysis. The emitter can generate a continuous light emission and the device includes a circuit that limits a time-averaged light from the emitter in a fashion that is independent from the controller. This circuit may be, for example, non-programmable.

In accordance with another embodiment of a device that provides an indication of visual system function of a patient, the device has an emitter capable of emitting visible light, an optical assembly arranged so that when in use light emitted from the emitter reaches an eye of the patient, and a controller. The controller when in use modulates a light emission from the emitter to create a light stimulus, receives and analyses an electrical signal from the visual system of the patient to create an analysis, and provides an indication of visual system function based on the analysis. The light stimulus comprises one or more flashes of light. The device further has a light detector arranged to detect light from the emitter, and a control circuit that modulates a duration of each flash of light based on an output from the light detector obtained during that flash of light.

In accordance with another embodiment, a method for providing an indication of visual system function of a patient includes illuminating an eye of the patient with a light stimulus from an emitter. Further, the method includes receiving and analyzing an electrical signal from the patient to create an analysis. The method also includes providing an indication of visual system function based on the analysis. The method also includes performing one or more of the following: controlling a temperature near the emitter; sensing the light stimulus with a detector and sensing the temperature near the detector; limiting a time-averaged light stimulus from exceeding a threshold using two or more independent circuits; and controlling the light stimulus, wherein the light stimulus comprises one or more flashes of light, by modulating a duration of each flash of light based on a light measurement obtained during that flash of light.

In some embodiments, the method includes more than one of the above-mentioned alternatives. The method may include controlling a temperature near the emitter and sensing the light stimulus with a detector and sensing the temperature near the detector. The method may include controlling a temperature near the emitter and limiting the time-averaged light stimulus using an independent circuit. The method may include sensing the light stimulus with a detector and sensing the temperature near the detector and limiting a time-averaged light stimulus from exceeding a threshold using two or more independent circuits. The method may include controlling a temperature near the emitter; sensing the light stimulus with a detector and sensing the temperature near the detector; and controlling the light stimulus, wherein the light stimulus comprises one or more flashes of light, by modulating a duration of each flash of light based on a light measurement obtained during that flash of light.

In accordance with another embodiment, a method for providing an indication of visual system function of a patient includes illuminating an eye of the patient with a light stimulus from an emitter. Further, the method includes receiving and analyzing an electrical signal from the patient to create an analysis. The method also includes providing an indication of visual system function based on the analysis. The method also includes controlling a temperature near the emitter.

In accordance with another embodiment, a method for providing an indication of visual system function of a patient includes illuminating an eye of the patient with a light stimulus from an emitter. Further, the method includes receiving and analyzing an electrical signal from the patient to create an analysis. The method also includes providing an indication of visual system function based on the analysis. The method also includes sensing the light stimulus with a detector and sensing the temperature near the detector.

In accordance with another embodiment, a method for providing an indication of visual system function of a patient includes illuminating an eye of the patient with a light stimulus from an emitter. Further, the method includes receiving and analyzing an electrical signal from the patient to create an analysis. The method also includes providing an indication of visual system function based on the analysis. The method also includes limiting a time-averaged light stimulus from exceeding a threshold using two or more independent circuits. One of those independent circuits does not have any programmable components.

In accordance with another embodiment, a method for providing an indication of visual system function of a patient includes illuminating an eye of the patient with a light stimulus from an emitter. Further, the method includes receiving and analyzing an electrical signal from the patient to create an analysis. The method also includes providing an indication of visual system function based on the analysis. The method also includes controlling the light stimulus, wherein the light stimulus comprises one or more flashes of light, by modulating a duration of each flash of light based on a light measurement obtained during that flash of light.

### BRIEF DESCRIPTION OF THE DRAWINGS

The accompanying drawings, which are incorporated in and constitute a part of this specification, illustrate several embodiments and together with the description, serve to explain the novel principles of the embodiments described herein. While different aspects of the invention are shown in different drawings, this separation is principally to improve the clarity; all aspects can be used in the same embodiment. In the drawings:

FIG. 1 is a schematic view of an exemplary visual electrophysiology device, highlighting certain aspects of the invention.

FIG. 2 is a schematic view of an exemplary visual electrophysiology device, highlighting certain aspects of the invention.

FIG. 3 is a schematic view of an exemplary visual electrophysiology device, highlighting certain aspects of the invention.

FIG. 4 is a schematic view of an exemplary visual electrophysiology device, highlighting certain aspects of the invention.

FIG. 5 is a flowchart diagram showing a method for providing an indication of visual system function of a patient.

### DETAILED DESCRIPTION

Disclosed herein are embodiments of improved visual electrophysiology devices and methods of improved visual electrophysiology. These devices and methods can be used to provide an indication of visual system function of a patient. These devices include an electrical circuit that controls a light stimulus directed toward the eye and measures the electrical signal the eye produces in response to the light. Device operation involves stimulating the eye with light and measuring an electrical response to the stimulus to create an analysis. By way of example, the analysis can be time span between the flash of light and the time of the peak of the electrical response, which may be indicative of the degree of retinal ischemia in a patient. Other analyses include various feature extractions from the electrical response, such as times and amplitudes of various features (e.g., a-wave, b-wave, PhNR), or more complex methods such as those involving wavelet analysis, logistic regressions, neural networks, machine learning, and the like. These analysis methods are known to those skilled in the art.

Embodiments of the present invention may improve the stimulus generation, for example, by improving the accuracy or consistency of the brightness of the stimulus, improving the consistency in the spectral characteristics of the stimulus, or by improving the optical safety of the device. The stimulus to the eye can comprise flashes of light or other modulated light waveforms. The stimulus to the eye can comprise a single flash of light. The stimulus to the eye can comprise a background illumination that is perceptually constant or only slowly changing.

Embodiments may provide an emitter capable of emitting visible light that may emit, for example, green, red, orange, blue, amber, yellow, or white light. Exemplary emitter types include an LED, laser diode, or xenon flashtube. Optionally, other (1, 2, 3, 4, 5, 6, 7, 8, 9, 10 or more) visible light emitters may be present with distinct or the same spectra. For example, some embodiments may use red, green, blue, and amber LEDs. Some embodiments may use multiple emitters having the same spectra to increase the brightness or increase the dynamic range of brightness. Some embodiments may have an infrared light emitter that emits at least 50% of its energy at wavelengths longer than 710 nm. Other emitters may be provided.

Embodiments may provide an optical assembly arranged so that light emitted from the emitter (when in use) reaches an eye of the patient. In some embodiments, the optical assembly provides diffuse light to the eye in order to stimulate the retinal generally. For example, the optical assembly may be a fraction of an integrating sphere or other diffuse reflecting surface. The RETeval device and UTAS Bigshot ganzfelds made by LKC Technologies are examples of optical assembles that are fractions of integrating spheres. The UTAS Sunburst ganzfeld made by LKC Technologies is an example of optical assembly that has a diffuse reflecting surface. Alternatively, the optical assembly can use lenses to provide diffuse light (e.g., using Maxwellian optics). Diffuse light is useful, for example, in full-field ERG and VEP measurements. In some embodiments, the optical assembly provides patterned or directed light to the eye. Patterned light is useful, for example, in pattern ERG, pattern VEP, multifocal ERG, multifocal VEP, and focal ERG measurements.

Some embodiments may use a controller to modulate a light emission from the emitter to create a light stimulus. For example, the light emission may be one or more flashes of light (e.g., flashes with a duration less than 6 ms, 21 ms, or 40 ms). By using pulse-width modulation (PWM) or other similar schemes known in the art, the apparent brightness of the light stimulus over time can be modulated so as to make stimuli that are dimmer or that are approximately sinusoidal, triangular, or rectangular. Individual flashes can have different luminance energies by changing the duration of the flash or by changing the instantaneous luminance of the flash. In embodiments having more than one emitter, the emission duration for each emitter may be different (e.g., a second emitter emits light for a longer period of time than the first emitter) or they may be the same.

In visual electrophysiology testing, the light stimulus is routinely changed in color or luminance to emphasize the response of different aspects of the visual pathway. Inadvertent changes in the light stimulus, e.g., due to intrinsic variability in the light source or due to variations in temperature of the emitter, may adversely impact the electrical signal measured from a visual system of the patient and therefore may adversely impact an indication of visual system function based on the measurement.

Some embodiments may use an active thermal control system configured to reduce temperature variability near the emitter. Light from an emitter may vary in both intensity and color as the temperature of the emitter changes. For example, LEDs generally have a reduced light emission as temperature increases due to increased recombination of electrons and holes that do not contribute to light emission while the emission color may change due to the temperature dependence of a semiconductor's bandgap. Thus, an active thermal control system may be useful in making a light stimulus from the emitter more consistent by reducing the temperature range experienced by the emitter. Other embodiments for reducing variability in the color of the emission use optical filters or use sources that are only minimally affected by temperature (e.g., laser diodes and xenon flashlamps). As an example, the active thermal control system configured to reduce temperature variability near the emitter may reduce a temperature range experienced by the emitter due to changing ambient temperature or due to self-heating of the emitter due to inefficiencies in the light generation process. This reduction in temperature range experienced by the emitter in turn may enable a more consistent light stimulus.

Some embodiments may use a light detector arranged to detect light from the emitter and a temperature sensor configured to detect a temperature near the light detector. As described in U.S. Pat. No. 9,492,098, the light detector may be used during a calibration phase of a test to compensate for variations in the output of the emitter or the optical efficiency of the optical assembly. However, not disclosed in the prior art is that further improvements can be made by appreciating the light detector's output may depend on temperature. By having temperature measurements near the light detector, the light detector's temperature dependence can be reduced. For example, the controller can use knowledge of the temperature dependence (obtained, e.g., from the product data sheet or from measurements) to correct the light detector's measurements. Alternatively, the temperature near the light detector can be actively controlled, for example, by using a heating element and a control circuit in addition to the temperature sensor. Having the temperature sensor close to the light detector improves the accuracy of the estimate of the light detector's temperature; the temperature sensor may be located such that the shortest distance between the temperature sensor and the light detector is less than 20 cm, 15 cm, 10 cm, 5 cm, 4 cm, 3 cm, 2 cm, 1 cm, or 0.5 cm from the light detector.

In certain cases, the desired light stimulus is a very bright but brief flash of light. For example, the ISCEV extended protocol for the stimulus-response series for light-adapted full-field ERG (McCulloch et al. (2019)) specifies the use of a 300 cd·s/m² luminance energy flash with a flash duration≤5 ms. If the flash was exactly 5 ms with a constant luminance, the luminance would be (300 cd·s/m²)/(5 ms)=60,000 cd/m². If this luminance was left on indefinitely, it may result in a light hazard to the retina. While some emitter types (e.g., xenon flashtubes) intrinsically stop emitting after a brief time period, other emitter types can provide a continuous light emission, for example, an LED and or a laser diode. Devices that use emitter types that can provide a continuous light emission therefore have some optical safety risk for generating a potential light hazard. Generally, the controller modulates a light emission from the emitter to create a light stimulus; however, in the event of an error condition in the controller (e.g., a software bug) having a circuit that limits a time-averaged light from the emitter in a fashion that is independent from the controller may reduce the optical safety risk. In addition to reducing the optical safety risk, the circuit may reduce the burden associated with developing software with a higher level of concern, especially if one of the two independent circuits (e.g., the controller and the independent circuit) does not have any programmable components.

Potential light hazards are described in detail in the international standard ISO 15004-2:2007. One such hazard is the retinal photochemical aphakic light hazard. One limit for this aphakic hazard is to keep a weighted retinal radiance L_{A-R} being below the limit of 2 mW/(sr cm²) when averaged over any 20 second interval. While L_{A-R} is carefully defined in ISO 15004-2:2007, briefly it sums the retinal radiance from the device at each wavelength after weighting the radiance by the aphakic photochemical hazard weighting function A(λ) which varies from 6 below 335 nm to 1.43 at 400 nm to 0.1 at 500 nm to 0.001 above 600 nm. Another such potential light hazard is the retinal visible and infrared radiation thermal hazard. One limit for this thermal hazard is to keep a weighted retinal visible and infrared radiation thermal radiance L_{VIR-R} below the limit of 6 W/(sr cm²) when averaged over any 20 second interval. While L_{VIR-R} is carefully defined in ISO 15004-2:2007, briefly it sums the retinal radiance from the device at each wavelength after weighting the radiance by the thermal hazard weighting function R(λ) which is 1 from 435 nm to 700 nm, tapering 0.2 for wavelengths≥1045 nm and tapering to 0 for wavelengths≤375 nm.

Some embodiments have a circuit may limit the time-averaged output of the emitter so that the device does not generate a potential light hazard. For example, the circuit may limit the time-averaged output of the emitter so that the device can be classified as a Group 1 instrument according to ISO 15004-2:2007, where no potential light hazards exist. For example, the circuit may limit a weighted retinal radiance L_{A-R} to a value no greater than 2 mW/(sr cm²) when averaged over any 20 second interval (e.g., a value of 0.1, 0.5, 1, or 2 mW/(sr cm²)). For example, the circuit may limit a weighted retinal visible and infrared radiation thermal radiance L_{VIR-R} to a value no greater than 6 W/(sr cm²) when averaged over any 20 second interval (e.g., a value of 0.1, 0.5, 0.6, 1, 2, 3, 4, 5, or 6 W/(sr cm²)). While ISO 15004-2:2007 uses a 20 second interval in the above calculations, other durations such as 30, 15, 10, 5, 4, 3, 2, 1, 0.5, 0.25, 0.1 seconds may also be used.

Some embodiments use a light stimulus comprising one or more flashes of light. In these cases, the device may have a light detector arranged to detect light from the emitter, and a control circuit that modulates a duration of each flash of light based on an output from the light detector obtained during that flash of light. One use of these embodiments is to provide a real-time luminance correction based on feedback from the light detector. For example, if the emitter's output has some brightness variability (e.g., the arcing characteristics in a xenon flashlamp or temperature-based variability), having the control circuit stop the light emission after the desired flash energy has been emitted may reduce the flash-to-flash luminance variability and thereby improve results from the device.

The above descriptions describe both a device providing an indication of visual system function of a patient as well as a method for providing an indication of visual system function of a patient.

Combinations of the above description are also contemplated. Composition and methods of their use are contemplated. Embodiments improve over existing visual electrophysiology devices in other ways apparent from the detailed description herein.

### Definitions

In order to provide more clear descriptions of the embodiments described herein, certain terms are defined as follows. Other terms are defined in other parts of this disclosure.

The term “emitter” refers to anything that emits electromagnetic radiation in the UV, visible, and infrared (IR) range. Exemplary emitters include LEDs, display devices, laser diodes, and gas-discharge devices such as xenon flash lamps and fluorescent bulbs. In some cases herein, the term “infrared” is abbreviated as “IR”.

The term “visible light” refers to electromagnetic radiation that can create a light stimulus. Visible light typically has a wavelength between 380 nm and 750 nm.

The term “light stimulus” refers to a visible light stimulus.

The term “LED” refers to a light emitting diode. LED includes those comprising semiconductor, organic, and quantum dots. The term LED includes those with integrated phosphors.

The term “patient” refers to a human or other vertebrate from which physiological electrical signals are to be measured. It is contemplated that the device will be placed in proximity to the patient to enable stimulation of the patient's visual system and measurement of physiological response thereto.

The phrase “indication of visual system function” refers to the analysis of an electrical signal from the visual system of a patient in response to light. It is to be distinguished from other measures of the visual system based solely on e.g., imaging of the eye structure with fundus photography, OCT, or the like, or psychophysical measures such as visual acuity using a Snellen chart.

The term “or” refers to inclusive or, where more than one (1) of the alternatives can be true.

The term “within” followed by a distance describing the relative locations of two objects refers to the shortest distance between the two objects. For example, if object A is within 3 cm of object B, then the shortest distance between object A and object B is less than or equal to 3 cm.

### DESCRIPTION

Various embodiments, as well as additional objects, features, and advantages thereof, will be understood more fully from the following description.

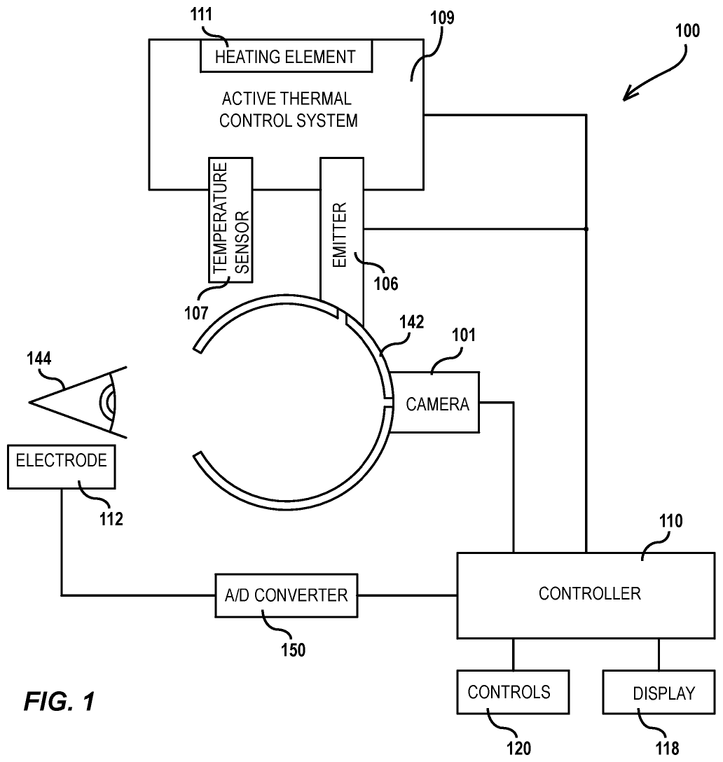

With reference to FIG. 1, shown is an exemplary device 100 used to provide an indication of visual system function of a patient. The emitter 106 shines light into an optical assembly 142, which when in use directs the light to the patient's eye 144. In this example, the optical assembly 142 acts as an integrating sphere to deliver the light emitted from the emitter 106 in a diffuse manner to the patient's eye. A diffuse light source enables interrogation of large portion of the retina and makes patient fixation less important. Optical assembly 142 may have a white interior surface to enhance the reflectivity. The white surface can be a coating (e.g., paint) or optical assembly 142 can be made for example, from white plastic. Other exemplary optical assemblies do not require light from the emitter 106 to be reflected before reaching the patient's eye, for example, the light may be refracted, diffused, scattered, or may have a direct path between the emitter and the patient's eye.

In some embodiments, emitter 106 may be an LED, laser diode, or a xenon flashlamp. More than one emitter may be used; for example, to provide greater luminance, a greater range in luminance, differing colors.

The emitter 106 can comprise 1, 2, 3, 4, or more emitters. For example, emitter 106 comprises a first emitter, which may be a LED or different type of light emitter. The first emitter has a first emission spectrum. In some embodiments, the first emitter may emit green, red, orange, blue, amber, white, or yellow light. The first emitter may be, for example, a green LED. Emitter 106 can also comprise a second emitter. The optional second emitter has a visible second emission spectrum that, for example, is distinct from the first emission spectrum. The second emitter, if present, may emit green, red, orange, blue, amber, white, or yellow light. The optional second emitter may be an LED or a different type of light emitter and may be, for example, a red LED. Emitter 106 can also comprise a third emitter. The optional third emitter has a visible third emission spectrum that, for example, is distinct from the first and second emission spectra. The third emitter, if present, may emit green, red, orange, blue, amber, white, or yellow light. The optional third emitter may be an LED or a different type of light emitter, and may be, for example, a blue LED. Emitter 106 can also comprise a fourth emitter. The optional fourth emitter has a visible fourth emission spectrum that, for example, is distinct from the first, second, and third emission spectra. The fourth emitter, if present, may emit green, red, orange, blue, amber, white, or yellow light. The optional fourth emitter may be an LED or a different type of light emitter, and may be, for example, an amber LED. The device 100 may have additional (e.g., 5, 6, 7, 8, or more) visible light emitters. Having four (4) different visible spectral sources enables independent stimulation of one of the three types of cones or rods in a human (Shapiro et al. (1996)).

Emitter 106 can be, for example, an RGB (red, green, blue) LED, for example, CREE CLVIL-FKB, CREE CLQ6A-FKW, CREE XLamp XML-L, Avago ASMT-MT000-0001, or Osram LRTD-C9TP. Emitter 106 can be, for example, an RGBA (red, green, blue, amber) LED, for example, CREE CLQ6A-YKW Individual LEDs or other light sources may be used, taken for example from the Luxeon C Color line LEDs or the Cree Xlamp XQ-E LEDs. The number of components in emitter 106 may be 1, 2, 3, 4, 5, 6, 7, 8, 9, 10, 11, 12 or more. A larger number of components comprising emitter 106 gives improved light uniformity in the integrating sphere and a brighter possible light output; however, larger numbers are inconvenient is terms of manufacturing difficulty and cost.

As shown in FIG. 1, camera 101 can image the patient's eye through the hole in optical assembly 142. Camera 101 may include an infrared light emitter; at least 50% of its energy will be emitted at wavelengths longer than 710 nm. The infrared light emitter can be used to illuminate the patient's eye during the exposure time of camera 101. In some embodiments, the device 100 has neither camera 101, nor an infrared light emitter.

As further components of device 100, the device 100 may include controls 120 that can be used to initiate each test and to enter customized settings. In addition, device 100 may have display 118 to assist the operator in using the device and for displaying test results.

Device 100 may receive an electrical signal from the visual system of the patient via electrode 112. Electrode 112 may be a disposable component, for example, as described in U.S. Pat. Nos. 9,510,762 and 10,010,261. Alternatively, electrode 112 may be a permanent component of device 100. Electrode 112, when used, may be near the eye, near the visual cortex on the back of the head, or in other locations selected so as to provide an electrical signal from the visual system of the patient. Electrode 112 may connect to an analog-to-digital (A/D) converter 150 that communicates with controller 110, which analyzes the data. Exemplary A/D converters include ADS1220, ADS1248, ADS1292, ADS1294, ADS1298, or ADS1299 from Texas Instruments and AD7195, AD7194, AD7193, AD7799, AD7738 from Analog Devices. Some embodiments do not use an A/D converter.

An embodiment of device 100 to provide an indication of visual system function of a patient has an emitter 106 capable of emitting visible light, an optical assembly 142 arranged so that when in use light emitted from the emitter reaches an eye 144 of the patient; and a controller 110. Controller 110 modulates a light emission from the emitter to create a light stimulus, receives and analyses an electrical signal from the visual system of the patient to create an analysis, and provides an indication of visual system function based on the analysis. Device 100 may also include an active thermal control system 109 configured to reduce a temperature variability near emitter 106, for example, within 0.5, 1, 2, 3, 4, or 5 cm of the emitter. Active thermal control system 109 may have a temperature sensor and at least one of a heater and a cooler to affect the temperature. The temperature sensor may a separate component (e.g., temperature sensor 107) located near emitter 106 (e.g., within 0.5, 1, 2, 3, 4, 5, 10, 15, or 20 cm of the emitter) or it may utilize measurements from a temperature-dependent aspect of emitter 106. For example, if emitter 106 is an LED, the voltage developed across the component at a certain current depends on temperature. When the LED is not being used, a small probe current could be used to generate the temperature-dependent voltage across the LED to be used by active thermal control system 109. Active thermal control system 109 may use a heater to increase the temperature near the emitter. The heater may be heating element 111. Heating element 111 may be a separate component located near emitter 106 (e.g., within 0.5, 1, 2, 3, 4, or 5 cm of the emitter). Heating element 111 may be a resistor or resistive traces on a printed circuit board. Alternatively, the heater may be emitter 106 as long as undesirable light emissions are avoided (e.g., with a shutter). Optionally, the active thermal control system includes a cooler to reduce the temperature. Having a heater and a cooler reduces the control logic's complexity, although it increases the complexity of the hardware (e.g., more components may be needed). The control logic for active thermal control system 109 may be part of controller 110 or it may be separate. The control logic may be a feedback type or it may include feedforward elements from the knowledge of upcoming power dissipation from the generation of a light stimulus. The active thermal control system may be configured to maintain a temperature near the emitter to 20° C., 25° C., 30° C., 35° C., 40° C., 45° C., 50° C., or hotter. Variations in the power dissipated in the emitter and variations in ambient temperature may cause variations in the emitter's temperature, leading to changes in the emission wavelength or luminance. This active thermal control system reduces the variations in the emitter's temperature thereby leading to improved stability of the emitted light.

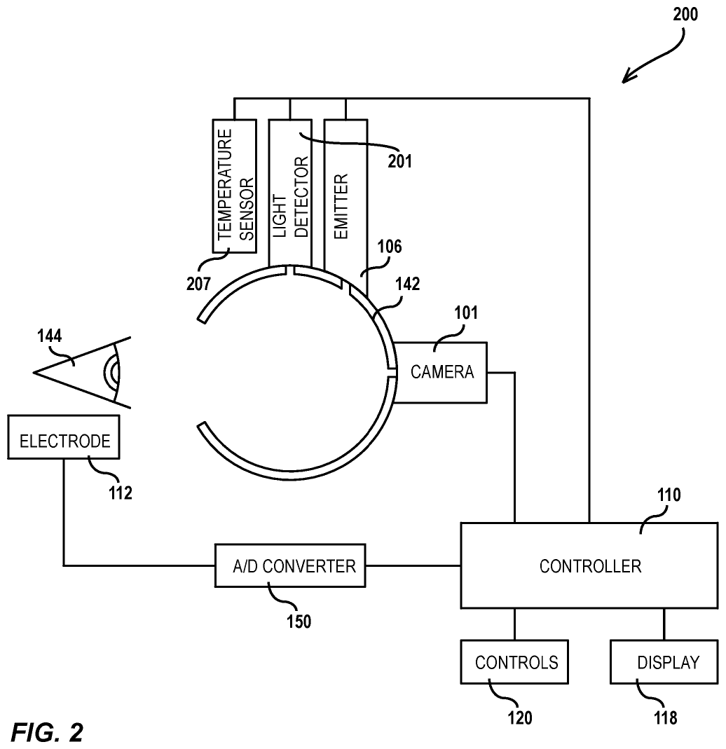

With reference to FIG. 2, device 200 may include a light detector 201 and temperature sensor 207. Temperature sensor 207 may be the same or different from optional temperature sensor 107 in FIG. 1. Light detector 201 may be arranged to detect light from emitter 106. Light detector 201 may be used to monitor the light stimulus so that the controller 110 can compensate for variations in the output of emitter 106 or in the optical efficiency of optical assembly 142. Controller 110 can adjust, for example during a calibration phase of a test, the output of emitter 106 in order to achieve a desired signal from light detector 105. If the adjustment is too great, the device 200 may be configured to report an error rather than possibly providing erroneous results. Temperature sensor 207 may be located within 0.5, 1, 2, 3, 4, 5, 10, 15, or 20 cm from light detector 201. Being close to the detector reduces any temperature differences between what is measured and the temperature of the light detector. Temperature measurements may be used along with a model of the temperature dependence of the light detector to reduce the temperature variability of the measurements. Alternatively, temperature measurements can be used in a light detector temperature control circuit to actively maintain the temperature. This light detector temperature control circuit may be the same or different from active thermal control system 109. If it is different, it may nevertheless have the same componentry: a sensor, a heater, optionally a cooler, and control logic.

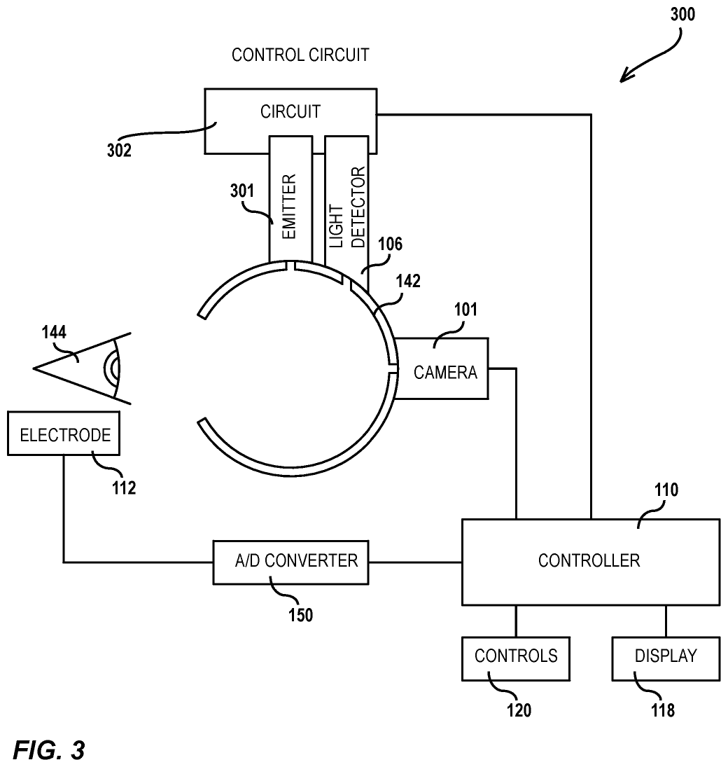

With reference to FIG. 3, device 300 may include circuit 302 that limits a time-averaged light from the emitter in a fashion that is independent from controller 110. In this way, the circuit 302 prevents the device 300 from generating a potential light hazard. Optional light detector 301 (which may be the same or different from optional light detector 201) measures the time-averaged light emission. This measurement can be compared to a threshold, where if exceeded, further light emission is stopped for a first time period, independent of controller 110.

Exemplary first time periods include fixed times such as 20 seconds or other durations such as 30, 15, 10, 5, 4, 3, 2, 1, 0.5, 0.25, 0.1 seconds. Other first time periods include the difference between above-mentioned exemplary time periods and the time taken to reach the desired time-averaged light limit. As a specific example, if the desired time-averaged light threshold was a weighted retinal radiance L_{A-R} of 2 mW/(sr cm²) when averaged over any 20 second interval, if in the first two seconds the retinal radiance was 20 mW/(sr cm²), this would be the maximum allowable retinal radiance over a 20 second time period so the circuit could prevent additional L_{A-R} for at least 18 seconds.

Methods to stop further light emission include actuating a shutter to block light from the emitter reaching the eye of a patient and preventing the emitter from generating light. Methods to prevent the emitter from generating light include interrupting its power supply (e.g., through high-side or low-side drivers) or by inserting a logic gate in the emitter's control circuit where both controller 110 and circuit 302 have to agree to turn on the emitter (e.g., if positive-logic is used in the emitter's control circuit, then an AND gate whose inputs are controller 110 and circuit 302 could connect to the emitter's control circuit).

Circuit 302 can be implemented using a programmable device or it can use only non-programmable components. The instantaneous light output from light detector 301 can be time-averaged digitally through a moving window or other low-pass filters known in the art. Alternatively, the instantaneous light output from light detector 301 can be time-averaged using analog electronics via a low pass filter.

An exemplary non-programmable implementation of circuit 302 follows. Light detector 301 may be a photodiode connected to a transimpedance amplifier that creates a voltage related to the light in optical assembly 142. This voltage can be filtered through an RC (or other type) low-pass filter and provided as an input to a comparator. When the comparator's voltage exceeds its set-point, it can deactivate emitter 106 using for example methods described above. The deactivation time can be set by a hardware one-shot (i.e., a monostable multivibrator) or a Schmitt-trigger feedback loop on the filtered voltage.

In cases where optional light detector 301 is not present, the power dissipated by, the voltage across, or current through emitter 106 can be used as a measurement of the light generated by emitter 106.

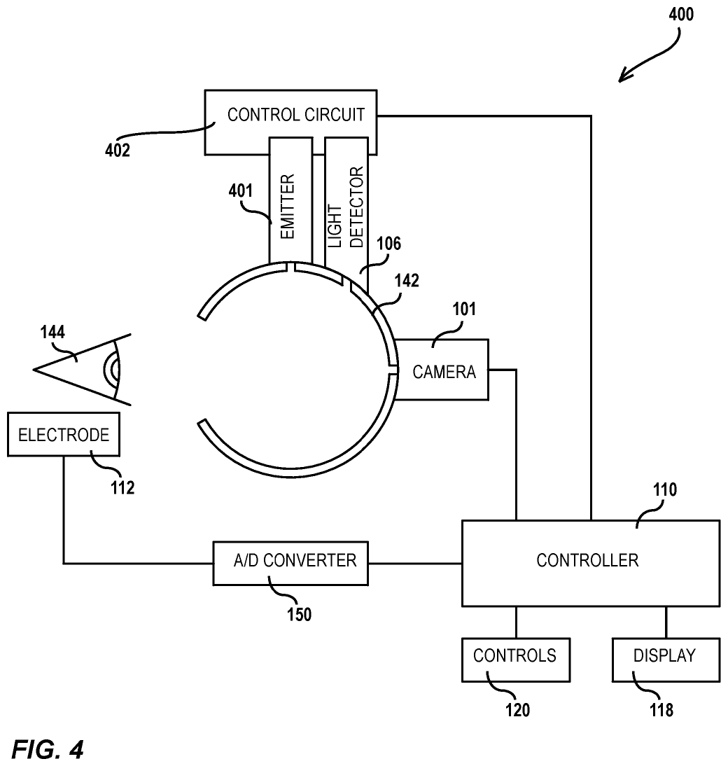

With reference to FIG. 4, device 400 may include control circuit 402. Light detector 401 (which may be the same or different from optional light detectors 201 and 301) measures the light emission. As described in U.S. Pat. No. 9,492,098, the light detector may be used during a calibration phase of a test to compensate for variations in the output of the emitter or the optical efficiency of the optical assembly. However, not disclosed in the prior art is using light detector 401 to adjust the flash duration (and therefore flash energy) in real-time. Because light flashes may be very short (e.g., <100 μs, <10 μs or <1 μs), measuring the flash energy and stopping the flash output when the desired flash energy is reached requires high-speed electronics. In some embodiments, a high-speed A/D converter (e.g. ≥100,000 samples per second or ≥1,000,000 samples per second) is used along with digital logic to determine when the proper amount of flash energy has been delivered and the flash should be stopped. In other embodiments, an integrator (or low-pass filter) circuit accumulates a signal related to the flash energy which is then sent to a comparator circuit. The comparator compares the signal to a target value, set for example, by a digital-to-analog converter (DAC). The DAC output can be set by controller 110. When the target value has been reached or exceeded, emitter 106 can be turned off by controller 110 or by using circuits described earlier.

Controller 110 provides the overall control of devices 100, 200, 300, and 400. Controller 110 may be a microcontroller or microcomputer device with a processor, memory and other connections to other components of device 100. Controller 110 may be a pre-programmed computer that is programmed to perform the functions and controls described herein. Alternatively, controller 110 may include wireless connections or wired connections that allow remote programming (e.g., for additional functions or updates). Those of ordinary skill in the art would understand how to program and operate controller 110. Control of emitter 106 is by means of controller 110 (and optionally the circuits describe above) which can control the timing of the light and camera sources, as well as the intensity, frequency and synchronicity thereof. By way of example, controller 110 can modulate the activity of the emitter 106, such as an LED to provide a series of brief flashes of light of predetermined duration, however, other stimulus waveforms or stimulus frequencies can also be utilized. Controller 110 can be a single microprocessor, for example, one sold by Analog Devices, Atmel, Intel, Microchip, Texas Instruments, etc. Alternatively, controller 110 can be distributed among many integrated circuits on one or more printed circuit boards in device 100. Controller 110 can be configured to modulate the light output of an emitter and to receive and analyze the electrical signal from a patient.

The analysis of the data from the electrical signals sensed by electrode 112 is carried out by controller 110. Algorithms for specifically assessing an indication of visual system function in a patient have been published. See, for example, Severns et al. (1991), Severns and Johnson (1991), Kjeka et al. (2013), and U.S. Pat. No. 9,931,032. Other algorithms are described in the references cited in the Background section above.

In some embodiments, controller 110 can communicate with a camera and measure the pupil size in images taken with the camera. In some embodiments, controller 110 can modulate the light output from additional emitters, such as a second emitter, a third emitter, a fourth emitter, or an infrared light emitter. Controller 110 can provide to the operator an indication for visual system function of a patient using a display and/or providing a means to communicate the information to a computer or other electronic device.

In some embodiments, the device includes more than one of the above-mentioned alternatives. For example, the device may include both an active thermal control system configured to reduce temperature variability near the emitter, and a light detector arranged to detect light from the emitter and a temperature sensor configured to detect a temperature near the light detector. As another example, the device may include an active thermal control system configured to reduce temperature variability near the emitter, an emitter that can generate a continuous light emission, and a circuit that limits a time-averaged light from the emitter in a fashion that is independent from the controller. As another example, the device may include a light detector arranged to detect light from the emitter and a temperature sensor configured to detect a temperature near the light detector, an emitter that can generate a continuous light emission, and a circuit that limits a time-averaged light from the emitter in a fashion that is independent from the controller. As another example, the device may include an active thermal control system configured to reduce temperature variability near the emitter, an emitter that can generate a continuous light emission, and a circuit that limits a time-averaged light from the emitter in a fashion that is independent from the controller, a light detector arranged to detect light from the emitter and a temperature sensor configured to detect a temperature near the light detector, and a circuit that limits a time-averaged light from the emitter in a fashion that is independent from the controller. As another example, the light stimulus comprises one or more flashes of light and the device may include a light detector arranged to detect light from the emitter, a temperature sensor configured to detect a temperature near the light detector, and a control circuit that modulates the durations of the light flashes based on an output from the light detector.

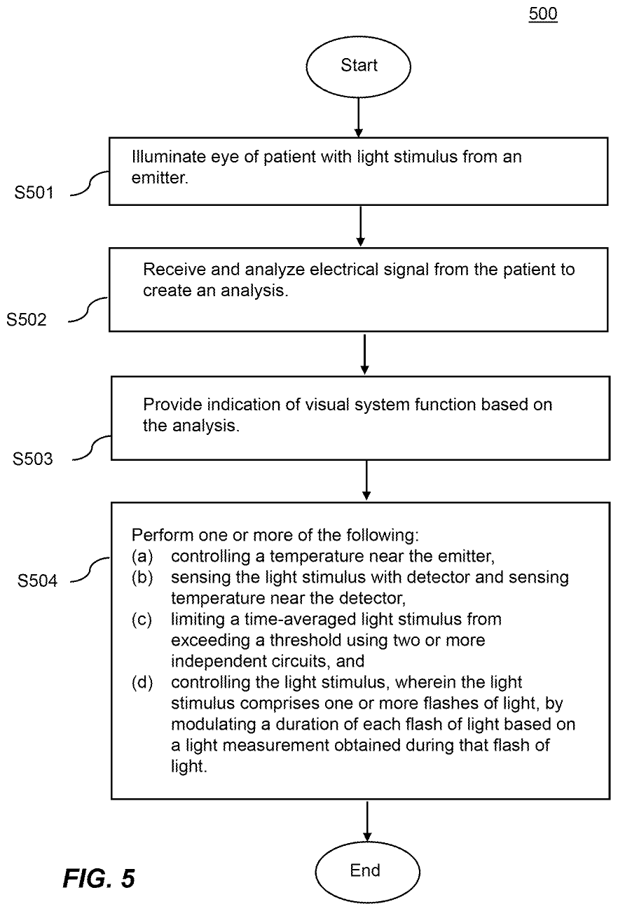

With reference to FIG. 5, shown is a flowchart diagram describing method 500 for providing an indication of visual system function of a patient. The method includes steps of illuminating an eye of the patient with a light stimulus from an emitter, block S501; receiving and analyzing an electrical signal from the patient to create an analysis, block S502; providing an indication of visual system function based on the analysis, block S503; and performing one or more of the following, block S504: (a) controlling a temperature near the emitter, (b) sensing the light stimulus with a detector and sensing a temperature near the detector, (c) limiting a time-averaged light stimulus from exceeding a threshold using two or more independent circuits, and (d) controlling the light stimulus, wherein the light stimulus comprises one or more flashes of light, by modulating a duration of each flash of light based on a light measurement obtained during that flash of light.

While the above descriptions have emphasized compositions, methods of their use are also contemplated. To provide an indication of visual system function, the methods involve illuminating an eye of the patient with a light stimulus. The methods also involve either receiving and analyzing an electrical signal from the patient so as to provide an indication of visual system function.

Some methods include techniques to reduce erroneous variations in the light stimulus. These techniques include controlling a temperature near the emitter. Both the color and intensity of light from the emitter may vary with temperature, so reducing the range of temperatures experienced by the emitter reduces variations in the light stimulus. In some embodiments, variations in the brightness of the light stimulus can be reduced using a light detector to monitor the light output. However, measurements from light detectors also vary with temperature. Accordingly, some methods include sensing the light stimulus with a detector and sensing a temperature near the detector. Sensing the temperature near the light detector can be employed to reduce the detector error by either mathematically adjusting the data knowing the temperature or by actively controlling the temperature near the detector. In some cases, it may be preferable to use LEDs or laser diodes to create brief flashes of light rather than using xenon flashlamps. However, LEDs and laser diodes can be inadvertently left on for long periods of time leading to inadvertently long exposures of light for the patient. Depending on the brightness of the emitter, this may create a potential light hazard. Accordingly, some methods include limiting a time-averaged light stimulus from exceeding a threshold using two or more independent circuits (for example, controller 110 and circuit 302). Having an independent circuit limiting the time-averaged light stimulus that is separate from the normal method of controlling the light stimulus may improve the overall safety of the device by requiring two independent failures to create the potential light hazard (the threshold being set so as to prevent the potential light hazard). Having at least one of independent circuits non-programmable may reduce the software development complexity by not having software affect optical safety. In some embodiments, a light detector is used to measure and modulate the durations of light flashes to reduce flash-to-flash variability. Accordingly, some methods include controlling the light stimulus, wherein the light stimulus comprises one or more flashes of light, by modulating a duration of each flash of light based on a light measurement obtained during that flash of light.

All references cited herein are incorporated by reference in their entirety. To the extent publications and patents or patent applications incorporated by reference contradict the disclosure contained in the specification, the specification is intended to supersede and/or take precedence over any such contradictory material.

All numbers expressing quantities used in the specification and claims are to be understood as being modified in all instances by the term “about.” Accordingly, unless indicated to the contrary, the numerical parameters set forth in the specification and attached claims are approximations that can vary depending upon the desired properties sought to be obtained by the present invention. At the very least, and not as an attempt to limit the application of the doctrine of equivalents to the scope of the claims, each numerical parameter should be construed in light of the number of significant digits and ordinary rounding approaches.

The above description of devices includes many novel and advantageous aspects. Combinations of the aspects are also contemplated. It will be apparent to those skilled in the art that various modifications and variations can be made in the disclosed detection device, components, and methods without departing from the scope of the invention. Other embodiments of the invention will be apparent to those skilled in the art from consideration of the specification and practice of the invention disclosed herein. It is intended that the specification and examples be considered as exemplary only, with a true scope of the invention being indicated by the following claims and their equivalents.

The terms and descriptions used herein are set forth by way of illustration only and are not meant as limitations. Those skilled in the art will recognize that many variations are possible within the spirit and scope of the invention as defined in the following claims, and their equivalents, in which all terms are to be understood in their broadest possible sense unless otherwise indicated.

### CITED REFERENCES

Bresnick, George, and Mari Palta. (1987) “Temporal Aspects of the Electroretinogram in Diabetic Retinopathy.” Arch Opthalmol, 105:660-4.
Han, Young-Keun, and Young-Hoon Ohn. (2000) “Changes of ERG Parameters in Diabetic Retinopathy.” J Korean Ophthalmol Soc, 41:149-155.
Bach, M, et al. (2012) “ISCEV standard for clinical pattern electroretinography—2012 update.” Doc Ophthalmol, 126:1-7.
Hoffman, M B, et al. (2012) “ISCEV standard for clinical multifocal electroretinography (mfERG) (2021 update).” Doc Ophthalmol, 142:5-16.
Kjeka, O, R W Jansson, C Bredru, and J Krohn. (2013) “Early panretinal photocoagulation for ERG-verified ischaemic central retinal vein occlusion.” Acta Ophthalmol, 37-41.
Odom, Vernon, et al. (2016) “ISCEV standard for clinical visual evoked potentials. (2016 update).” Doc Ophthalmol, 133:1-9.
Satoh, S, H lijma, M Imai, K Abe, and T Shibuya. (1994) “Photopic electroretinogram implicit time in diabetic retinopathy.” Japanese Journal of Ophthalmology, 38:178-184.
Severns, M L, Johnson, M A and Merritt, S A. (1991) “Automated estimate of implicit time and amplitude of the flicker electroretinogram.” Applied Optics 30:2106-2112.
Severns, M L and Johnson, M A (1991) “Automated implicit time and amplitude determination for the 30 Hz flicker electroretinogram: performance prediction of neovascularization central retinal vein occlusion.” Technical Digest Series, Washington, D.C; Optical Society of America, pp. 10-13.
Shapiro, A G, Pokorny J, and Smith V C. (1996). “Cone-rod receptor spaces with illustrations that use CRT phosphor and light-emitting-diode spectra.” J. Opt Soc. Am. A., 2319-2323.
Robson, A G, et al. (2022) “ISCEV standard for full-field clinical electroretinography (2022 update).” Doc Ophthalmol.

1: A device providing an indication of visual system function of a patient comprising:
a. an emitter capable of emitting visible light;
b. an optical assembly arranged so that light emitted from the emitter reaches an eye of the patient; and
c. a controller configured to:
(i) modulate a light emission from the emitter to create a light stimulus,
(ii) receive and analyze an electrical signal from a visual system of the patient to create an analysis, and
(iii) provide an indication of visual system function based on the analysis,
wherein the device is further configured to provide one or more conditions selected from the group consisting of:
(i) the device further comprises an active thermal control system configured to reduce temperature variability near the emitter;
(ii) the device further comprises a light detector arranged to detect light from the emitter and a temperature sensor configured to detect a temperature near the light detector;
(iii) the emitter is capable of providing a continuous light emission and the device further comprises a circuit that limits a time-averaged light from the emitter in a fashion that is independent from the controller; and
(iv) the light stimulus comprises one or more flashes of light, the device further comprises a light detector arranged to detect light from the emitter, and the device further comprises a control circuit that modulates a duration of each flash of light based on an output from the light detector obtained during that flash of light.

2: The device of claim 1 wherein the emitter is selected from an LED, a laser diode, or a xenon flashlamp.

3: The device of claim 1 wherein the device comprises an active thermal control system configured to reduce temperature variability near the emitter.

4: The device of claim 1 wherein the device further comprises a light detector arranged to detect light from the emitter and a temperature sensor configured to detect a temperature near the light detector.

5: The device of claim 1 wherein the emitter is capable of providing a continuous light emission and the device further comprises a circuit that limits a time-averaged light from the emitter in a fashion that is independent from the controller.

6: The device of claim 1 wherein the light stimulus comprises one or more flashes of light, the device further comprises a light detector arranged to detect light from the emitter, and the device further comprises a control circuit that modulates a duration of each flash of light based on an output from the light detector obtained during that flash of light.

7: The device of claim 1 wherein the emitter is capable of providing a continuous light emission and the device further comprises:
an active thermal control system configured to reduce temperature variability near the emitter;
a light detector arranged to detect light from the emitter and a temperature sensor configured to detect a temperature near the light detector; and
a circuit that limits a time-averaged light from the emitter in a fashion that is independent from the controller.

8: The device of claim 3 wherein the active thermal control system uses an electrical property of the emitter to infer its temperature.

9: The device of claim 8 wherein the emitter is a light emitting diode (LED) and the electrical property is a voltage across the LED resulting from a current flowing through the LED.

10: The device of claim 3 wherein the active thermal control system comprises
a temperature sensor located such that the shortest distance between the temperature sensor and the emitter is less than 3 cm; and
a heating element located such that the shortest distance between the heating element and the emitter is less than 3 cm.

11: The device of claim 10 wherein the active thermal control system is configured to maintain a temperature near the emitter to a value above 25° C.

12: The device of claim 4 wherein the temperature sensor is located such that the shortest distance between the temperature sensor and the light detector is less than 3 cm.

13: The device of claim 12 wherein the controller uses a measurement from the light detector to adjust the light emission.

14: The device of claim 13 wherein the controller uses a measurement from the temperature sensor to adjust the light emission.

15: The device of claim 13 further comprising a heating element and a control circuit configured to reduce the temperature variability near the light detector.

16: The device of claim 5 wherein the circuit limits the time-averaged light from the emitter so as to prevent the device from generating a potential light hazard.

17: The device of claim 16 wherein the potential light hazard is defined as a weighted retinal radiance L_{A-R} being greater than 2 mW/(sr cm²) when averaged over any 20 second interval.

18: The device of claim 5 wherein the circuit does not contain any programmable components.

19: The device of claim 7 wherein
(a) the active thermal control system comprises
a temperature sensor located such that the shortest distance between the temperature sensor and the emitter is less than 3 cm; and
a heating element located such that the shortest distance between the heating element and the emitter is less than 3 cm;
(b) the active thermal control system is configured to maintain a temperature near the emitter to a value above 25° C.;
(c) the active thermal control system is further configured to maintain a temperature near the light detector to a value above 25° C.;
(d) the temperature sensor is additionally located such that the shortest distance between the temperature sensor and the light detector is less than 3 cm;
(e) the controller uses a measurement from the light detector to adjust the light emission; and
(f) wherein the circuit limits the time-averaged light from the emitter so that the device can not generate a potential light hazard, the potential light hazard defined as a weighted retinal radiance L_{A-R} being greater than 2 mW/(sr cm²) when averaged over any 20 second interval.

20: The device of claim 6, wherein the control circuit comprises a digital to analog converter, an integrator circuit, and a comparator circuit, wherein:
(a) the controller is configured to set an analog setpoint using the digital to analog converter;
(b) the output from the light detector is integrated using the integrator circuit to generate a flash energy signal;
(c) the comparator circuit is configured to compare the analog setpoint with the flash energy signal; and
(d) an output from the comparator circuit is used to stop the light emission, thereby modulating the duration of the light flashes.

21: The device of claim 20, wherein the emitter is a xenon flashlamp.

22: A method for providing an indication of visual system function of a patient, comprising:
illuminating an eye of the patient with a light stimulus from an emitter;
receiving and analyzing an electrical signal from the patient to create an analysis;
providing an indication of visual system function based on the analysis; and
further performing one or more of the following steps selected from the group consisting of:
(a) controlling a temperature near the emitter,
(b) sensing the light stimulus with a detector and sensing a temperature near the detector,
(c) limiting a time-averaged light stimulus from exceeding a threshold using two or more independent circuits, and
(d) controlling the light stimulus, wherein the light stimulus comprises one or more flashes of light, by modulating a duration of each flash of light based on a light measurement obtained during that flash of light.

23: The method of claim 22 wherein the temperature near the emitter is controlled.

24: The method of claim 22 wherein a detector is used to sense the light stimulus and a temperature near the detector is measured.

25: The method of claim 22 wherein the time-averaged light stimulus is limited from exceeding a threshold using two or more independent circuits.

26: The method of claim 22 wherein the light stimulus comprises one or more flashes of light and a duration of each flash of light is modulated based on a light measurement obtained during that flash of light.

27: The method of claim 22 wherein the temperature near the emitter is controlled, the time-averaged light stimulus is limited from exceeding a threshold using two or more independent circuits, and a detector is used to sense the light stimulus and a temperature near the detector is measured.

28: The method of claim 23 wherein an electrical property of the emitter is used to infer its temperature.

29: The method of claim 28 wherein the emitter is a light emitting diode (LED) and the electrical property is the voltage across the LED resulting from a current flowing through the LED.

30: The method of claim 23 wherein the temperature is controlled using
a temperature sensor located within 3 cm of the emitter; and
a heater located within 3 cm of the emitter.

31: The method of claim 30 wherein the temperature is controlled to a value above 25° C.

32: The method of claim 24 wherein the temperature measured is within 3 cm of the detector.

33: The method of claim 32 wherein a light emitted from the emitter is adjusted based on an output of the detector.

34: The method of claim 33 wherein the light emitted is adjusted based on the temperature measurement.

35: The method of claim 33 wherein the temperature is controlled near the emitter.

36: The method of claim 25 wherein the time-averaged light stimulus is limited so as to prevent a potential light hazard from being generated.

37: The method of claim 36 wherein the potential light hazard is defined as a weighted retinal radiance L_{A-R} being greater than 2 mW/(sr cm²) when averaged over any 20 second interval.

38: The method of claim 37 wherein at least one of the independent circuits does not contain any programmable components.

39: The method of claim 27 wherein:
(a) the temperature is controlled using a temperature measured within 3 cm of the emitter and within 3 cm of the detector; and a heater located within 3 cm of the emitter and within 3 cm of the detector,
(b) the temperature is controlled to a value above 25° C.,
(c) a light emitted from the emitter is adjusted based on an output of the detector, and
(d) the time-averaged light stimulus is limited so that the device can not generate a potential light hazard, the potential light hazard is defined as a weighted retinal radiance L_{A-R} being greater than 2 mW/(sr cm²) when averaged over any 20 second interval.

40: The method of claim 26 wherein the emitter is a xenon flashlamp.
